(19) United States
(12) Patent Application Publication
(10) Pub. No.: US 2026/0112471 A1
(43) Pub. Date: Apr. 23, 2026

(54) STREAMLINING FULFILLMENT OF PRESCRIPTIONS SUBJECT TO ELEVATED REGULATION

(71) Applicant: Flamel Ireland Limited, Dublin (IE)

(72) Inventors: Robert Chuba, Carlsbad, CA (US); Danielle Suzanne Burroughs, Ballwin, MO (US); Meggan Curta Sullivan, Downers Grove, IL (US); Joseph V. Fenton, Flemington, NJ (US)

(21) Appl. No.: 19/361,943

(22) Filed: Oct. 17, 2025

Related U.S. Application Data
(60) Provisional application No. 63/708,466, filed on Oct. 17, 2024.

(51) Int. Cl. G16H 20/10; G06F 40/174; G16H 10/60

(52) U.S. Cl. CPC G16H 20/10; G06F 40/174; G16H 10/60

(57) ABSTRACT

A fulfillment system includes a platform for facilitating fulfillment of a prescription for a regulated product, such as a prescription drug, which also has a REMS, along with any applicable controlled substance scheduling requirements. The platform communicates with a computer network of a healthcare provider's office where a healthcare provider or other authorized prescriber issues a prescription for a patient. The platform also communicates with a pharmacy, such as a specialty pharmacy, for fulfillment of the prescription. The system electronically populates documents satisfying documentation requirements pertaining to a REMS, controlled substances regulations, prescriptions, other pertinent patient information, and/or other regulations or rules thereby eliminating paper and redundant documents and reducing the burden on the healthcare provider/healthcare system.

### CROSS REFERENCE TO RELATED APPLICATION

This application claims priority to U.S. Provisional Application No. 63/708,466, filed Oct. 17, 2024.

### FIELD OF THE INVENTION

The present invention relates generally to fulfillment of prescriptions for drugs. More particularly, the invention relates to a system and associated methodology for simplifying compliance with multiple documentation requirements for prescriptions subject to elevated regulation. The documentation requirements may be requirements associated with state or federal regulations, documentation requirements of parties involved in the fulfillment process, or other sources of documentation requirements.

### BACKGROUND OF THE INVENTION

Many countries, states, and other jurisdictions regulate access to specified products including certain drugs. Typically, access to drugs is regulated to prevent abuse/misuse/diversion, to ensure that drugs are being used properly, and to avoid potentially dangerous drug interactions or other serious adverse outcomes, among other things.

Some drugs are subject to elevated regulation due to a perceived heightened need to monitor proper use. In the United States, drugs may be classified as over-the-counter (OTC) drugs, which are subject to a lower level of regulation, and prescription drugs. Prescription drugs require a prescription, written by an authorized prescriber (e.g., a healthcare provider, such as a physician) to a pharmacy and are limited to use by an identified person for a prescribed use. There is a significant body of regulations covering the issuance and fulfillment of prescriptions.

In addition, some drugs are subject to other regulations. In the United States, drugs may be regulated under a number of overlapping federal and state regimes. Under federal law, some drugs are subject to the Comprehensive Drug Abuse Prevention and Control Act of 1970. Title II of this Act is known as the Controlled Substances Act (CSA). The United States Drug Enforcement Administration is primarily responsible for implementing and enforcing the CSA. Under the CSA, the federal government pursues the objectives of making access to drugs available for legitimate medical purposes while protecting the public by preventing diversion of the drugs into illicit markets. To those ends, the CSA identifies drugs subject to control and establishes five schedules of controlled substances. Schedule I drugs are subject to the strictest controls, while Schedule V drugs have the lowest level of controls. Drugs are assigned to these schedules based on consideration of their medical utility as well as the potential for abuse and potential to create physical and psychological dependency. The CSA primarily applies to prescription drugs, not OTC drugs. A prescription drug may also be a controlled substance.

In the United States, drugs are also subject to regulation under the federal Food, Drug, and Cosmetic Act (FD&C Act). The United States Food and Drug Administration (FDA) is primarily responsible for enforcing this Act. This Act protects consumers against unsafe or ineffective drugs. Among other things, the FDA protects against drugs that are misbranded or adulterated. Prescription drugs the FDA has not approved are considered misbranded. The FDA also regulates labeling, advertising, and promotion of drugs.

In addition to these federal laws, drugs may be subject to state laws. The state laws may regulate drugs differently than regulated by the federal government or may place additional regulations on drugs subject to federal regulations. Thus, certain drugs are subject to both state and federal regulation.

The CSA, FD&C Act, and state laws are thus separate and complementary regimes. Any person or organization that produces, fulfills, or otherwise works with prescription drugs that are also state and federal controlled substances must comply with the CSA, the FD&C Act, and each individual state's laws. In addition, such a person or entity must comply with the regulations concerning prescription drugs, among other things.

Additional requirements may be imposed by non-government actors. For example, a drug may be designated as a specialty medication subject to a specialty pharmacy mandate. Such a designation may be due to price, special handling requirements, or because the drug is only available via a limited fulfillment network. The designation may be made by a manufacturer or other drug applicant holder (“applicant holder”), an insurer or payor, or other prescription management entity. A specialty pharmacy mandate is a set of rules generally made by insurance companies or other payors. Under these rules, a patient must get certain medications from certain specialty pharmacies. These specialty pharmacies may be owned by pharmacy benefit managers or independently owned. Applicant holders sometimes rely on specialty pharmacies to report patient status, reasons for termination of treatment, and other matters.

Drug fulfillment may further involve patient support services. Patient support services are intended to provide care coordination and provide education and more comprehensive patient services, among other things. The services may include benefits investigations, prior authorization, education, drug delivery and administration coordination, financial and co-pay assistance, patient education, data reporting, and administering branded support programs. Different service providers may provide different services and combinations of services in this regard. For convenience, a service platform that provides any or all of these services, in a REMS or other fulfillment context, may be referred to as a hub. Use of hub services may be voluntary, or it may be mandated. Although the services provided by a hub and a specialty pharmacy can overlap, a hub can help standardize patient care and education on support access to the fulfillment network including the specialty pharmacies. Hub services may involve multiple participating specialty pharmacies, for example, because individual specialty pharmacies may only have contracts with certain payors.

Some drugs in the United States are subject to a Risk Evaluation and Mitigation Strategy (REMS) promulgated by the FDA. Such a REMS may specify, for example, that the applicant holder must provide certain information to patients and healthcare providers, patients must be enrolled in the REMS, healthcare providers must be enrolled and certified in the REMS, fulfillment is restricted to certain specialty pharmacies certified in the REMS, and/or the drug is only administered in REMS-certified healthcare facilities with observation requirements. A given drug may be subject to both prescription regulations and REMS regulations.

These regulations and other rules typically include documentation requirements. Prescription regulations, for example, require a prescription signed by an authorized healthcare provider. There may be a REMS prescription form as well as other required forms. In some REMS, healthcare providers, pharmacies and/or others involved in fulfillment have to complete required forms. For example, healthcare providers may be required to enroll and be certified in a REMS and attest that they will follow FDA REMS requirements. In addition, REMS administrators, specialty pharmacies, hubs, and/or others may require enrollment and other documentation that is not necessarily required by government regulations. While these documentation requirements help ensure proper control of access to and safe use of the regulated drugs, they can be cumbersome and redundant to the healthcare system and interfere with or delay access to drugs needed by patients.

### SUMMARY OF THE INVENTION

The present invention is directed to a system and associated functionality to facilitate a fulfillment process for drugs subject to elevated regulation. The invention simplifies compliance with multiple sets of documentation requirements. For example, the documentation requirements may relate to regulations for prescriptions, a REMS, controlled substances, or other regulatory framework, and/or may have document requirements of parties involved in the fulfillment process. The invention allows for use of a single collection of patient information to populate forms for compliance with different documentation requirements including populating prescription forms, hub forms, REMS enrollment forms, and/or other required forms. The patient information can also be used to populate forms required for action to be initiated in the process such as action to be taken in the pharmacy used for fulfillment. In this manner, the fulfillment process is streamlined while maintaining appropriate compliance with applicable regulations required for access. In addition, appropriate and timely access to regulated drugs is supported while paper documentation is reduced or simplified.

In accordance with one aspect of the present invention, a system and associated functionality (“utility”) is provided whereby a single set of patient information can be used to electronically populate documents for separate documentation requirements, including documents required by separate, independently managed entities/destinations. The utility involves receiving patient information including data corresponding to fields of information concerning REMS enrollment, a REMS prescription form, hub enrollment, patient support services, or other documentation. For example, the data may include information identifying the patient, the drug, and the prescribing healthcare provider, among other things. This information may be entered at a healthcare provider's office or via a user device (e.g., a laptop computer, a tablet computer, or other mobile device), for example, using logic resident on a computer network of the healthcare provider and/or logic accessed via a remote computer network platform. The information may be obtained from an Electronic Health Record (EHR)/Electronic Medical Record (EMR) system and/or entered at any appropriate site of the fulfillment network.

This information may then be processed locally on a system of the healthcare provider and/or at a separate computer network platform to perform certain functions. These functions include extracting, from the patient information, data corresponding to a first set of defined fields, using the data to populate data objects of a document that at least partially satisfies documentation requirements (e.g., of a regulatory framework or a party involved in fulfillment), and providing the document(s) to a party involved in fulfillment of the prescription. Depending on the document and the associated REMS or other requirements, the document may be provided electronically, by fax (e.g., via a fax server), by mail, or other appropriate delivery mode. Via a similar process, data can be extracted from the patient information and used to populate another required document, e.g., a REMS enrollment document, and the document may be provided to a party involved in fulfillment such as a REMS Administrator managing patient REMS enrollments. Two or more separate documents for prescription fulfillment are thus generated electronically from a single set of patient information thereby simplifying compliance with regulations or other documentation requirements and decreasing the burden on the healthcare provider.

This can be implemented in a variety of ways and in a variety of contexts. In one implementation, the documents may relate to REMS requirements and to prescription documentation requirements (e.g., in the case of a required REMS prescription form). For example, the documents may relate to an enrollment form of a patient support services program, a REMS prescription form, and/or another form required as part of a REMS. Moreover, the patient information may be used to generate similar forms for successive events such as regenerating prescription documentation when a pharmacy changes. In this regard, the data extracted for the first and second documents may be the same, different, or partially overlapping. Depending on the context, the forms may be transmitted to a healthcare provider, to a fulfillment pharmacy such as a specialty pharmacy, a REMS Administrator, or another responsible party. It will be appreciated that regulations generally require the prescription be transmitted directly from the healthcare provider to the pharmacy. In cases where the documentation requirements are regulatory, the regulatory requirements may be separate requirements of a single regulatory framework, such as a REMS, or may involve separate regulatory frameworks, such as REMS, controlled substance, and/or prescription regulations.

The inventive functionality may be implemented in whole or in part on a computer network platform, e.g., an Internet platform such as a cloud-based platform. The platform may communicate with computer networks of a healthcare provider, a pharmacy, REMS Administrator, or other parties. In this regard, the communications between the platform and other computers/networks are compliant with applicable regulations concerning prescriptions and privacy, e.g., the Health Insurance Portability and Accounting Act of 1996, as may be amended from time to time, and the regulations promulgated thereunder (HIPAA), prescription regulations, and any applicable REMS requirements. In addition, the inventive functionality may involve configuring data in relation to a number of defined fields relating to the patient, the drug involved, the prescribing healthcare provider, insurance information, prescription assistance information, and the like. The patient information may be accessed via an EHR system and/or entered using a template that prompts the user to enter items of information in response to prompts, e.g., questions or entries on user interface forms. These data items may be associated with appropriate metadata to facilitate use of the data to electronically populate fields of subsequent forms and other processing. The logic associated with generating templates, configuring the data, and controlling communications may be implemented on the platform, on the remote computers/networks of users, or distributed therebetween.

The invention thus facilitates fulfillment of prescriptions for drugs subject to elevated regulation. It enables documents to be electronically generated from a single set of patient information, obtained from an EHR/EMR source and/or entered by a user, thereby reducing redundant data entry, reducing or eliminating the need for certain paper documents, and reducing the burden on the healthcare provider and/or the healthcare system. The documents can be distributed to separately managed entities and may include information that is duplicative across the entities as well as information that is unique to a specific, independently managed entity. The invention also facilitates documentation in relation to changes such as changing a fulfillment pharmacy and may improve timely fulfillment of prescriptions in compliance with applicable regulations and other documentation requirements.

### BRIEF DESCRIPTION OF THE DRAWINGS

For a more complete understanding of the present invention and further advantages thereof, reference is now made to the following Detailed Description, taken in conjunction with the drawings, in which:

FIG. 1 is a schematic diagram of a fulfillment system in accordance with the present invention;

FIG. 2 is a flowchart showing a process associated with a fulfillment system in accordance with the present invention; and

FIG. 3 is a flowchart showing a process associated with a healthcare provider in accordance with the present invention.

### DETAILED DESCRIPTION

The present invention is directed to a system and associated functionality for facilitating fulfillment of prescriptions for drugs subject to elevated regulation. In the following description, the invention is set forth in the context of a system where a single set of patient information is used to generate documents, required by separately managed entities, associated with prescription regulations, controlled substance regulations, REMS requirements, and other requirements for fulfillment of a prescription. There are many drugs that require a prescription and are subject to a REMS and this represents a particularly advantageous application of the present invention. In such cases, the prescription may be required to be filled by a specialty pharmacy as in certain examples noted below. There are also many drugs that that use hub services in the fulfillment process. While the invention has particular advantages in these contexts, it will be appreciated that various aspects of the invention are not limited to these contexts or the implementations described in detail below. Accordingly, the following description should be understood as illustrative and not by way of limitation.

As noted above, in the United States, some drugs are subject not only to the regulations governing prescription drugs but also to regulations concerning controlled substances and a REMS specific to the drug or class of drugs. One example of this is oxybate drugs, including sodium oxybate, LUMRYZ™ or XYREM™, and XYWAV™ (calcium, magnesium, potassium & sodium oxybates), approved for the treatment of certain conditions (e.g., narcolepsy). All oxybate drugs are subject to a REMS intended to mitigate the risks of serious adverse outcomes resulting from inappropriate prescribing, misuse, abuse, and diversion. The REMS requires, among other things but not fully inclusive, prescriber enrollment and certification, patient enrollment, pharmacy enrollment and certification, patient counseling, and fulfillment via a certified specialty pharmacy. Documentation of, for example, the REMS enrollments, patient counseling, and shipments, among other required documentation, can be collected in one or more databases. In addition, compliance with the regulations concerning prescriptions is required. Those regulations require, among other things, that a signed REMS prescription form is transmitted directly from the healthcare provider to a certified specialty pharmacy. Such specialty pharmacies may be used in fulfillment processes for multiple drugs subject to multiple REMS.

In general, the process for prescribing one of these drugs begins with a patient visiting a healthcare provider's office for diagnosis and treatment. The healthcare provider, likely a specialist, initiates the prescription process and will be aware (or become aware) of the REMS requirements. Today, for these drugs, this generally requires that enrollment forms and other documentation required by the REMS be submitted and that a REMS prescription form be on file for each patient at the appropriate specialty pharmacy prior to dispensing. Some REMS require a prescription form in addition to other prescription requirements, such as information concerning a patient's other medications, comorbidities history, and other information pertinent to safe fulfillment. A single prescription form that satisfies the REMS, controlled substance, and prescription requirements may be utilized and meet all requirements for some states but not all states. This is a cumbersome process that may delay, interfere with, or complicate the fulfillment process.

The present invention provides a system and process for satisfying REMS, controlled substance, prescription, and potentially other documentation requirements via forms that are populated from a repository of patient information and electronically submitted. Certain REMS requirements may be satisfied via a document which is electronically completed, and a valid electronic signature is captured, and prescription requirements may be satisfied via an electronic prescription from the EHR or similar electronic platform of the healthcare provider (e.g., a portal, website, etc., containing an integrated e-prescribing tool such as the Surescripts system) where required by state regulations. In this manner, multiple REMS, controlled substance, prescription, and other requirements are satisfied without requiring “paper” or redundant submissions.

For example, in the system described herein, some or all of the following forms may be populated from a single repository of patient information:
1. A patient support services enrollment form such as a hub enrollment form(s);
2. A REMS patient enrollment form (this is generally a one-time process unless a patient were disenrolled from the REMS due to misuse, abuse, or diversion, in which case the patient would have to re-enroll if the HCP and applicant holder approved);
3. A REMS prescription form (e.g., for the initial dispense at each specialty pharmacy and if a patient has a 6-months or more lapse in drug treatment);
4. An electronic prescription (when required by state law); and
5. Any other enrollments, e.g., for the hub, specialty pharmacy, prescription assistance programs, and the like.

A number of different architectures are possible for implementation of the fulfillment system of the present invention. For example, healthcare providers typically use an EHR system for collecting and accessing medical records. Such EHRs may be embodied as a network platform that is accessed by providers to capture, store, and share medical information. The functionality of the present invention can be integrated, at least in part, into an EHR system. In this manner, providers can access a familiar system, patient information can be readily accessed for populating forms, and records can be saved to the EHR system. Alternatively, an EHR system, or certain functionality thereof, can be integrated into a separate platform, such as a hub. Again, this facilitates access to patient information and storage of records. As a further alternative, an EHR system and a fulfillment system can be provided as separate, but cooperating platform, not integrated into a hub, healthcare provider, REMS Administrator, specialty pharmacy, or other conventional fulfillment platform. In the description below, an example of a fulfillment system is described where certain functionality is integrated into a hub and additional functionality is distributed between the hub, a healthcare provider and its EHR, a REMS Administrator, and specialty pharmacy platforms. It will be appreciated that the functionality, including EHR functionality, may be provided using a different architecture.

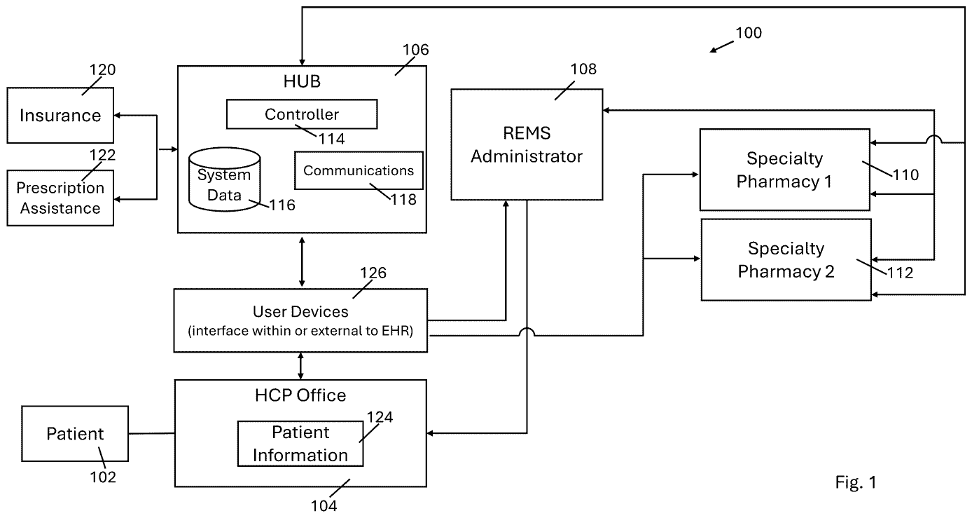

Referring to FIG. 1, a fulfillment system 100 in accordance with the present invention is shown. The system 100 includes hub platform 106 for facilitating fulfillment of a prescription for a regulated drug, such as a prescription drug, which also has a REMS. It will be appreciated that the hub platform 106 may provide hub services and other services. In addition, the hub services may be implemented at the platform and/or distributed between the platform and other platforms or system components. The platform 106 communicates with a computer network of a healthcare provider's office 104, e.g., a doctor's office, where a physician or other authorized prescriber issues a prescription for a patient 102. The platform 106 also communicates with a pharmacy, in this case a specialty pharmacy 110 or 112 for fulfillment of the prescription. Although two specialty pharmacies are shown for purposes of illustration, more specialty pharmacies may be part of the dispensing network. For example, in one implementation, four specialty pharmacies are involved, including three commercial specialty pharmacies and one noncommercial specialty pharmacy. In addition, the office 104 communicates with a REMS Administrator 108 (e.g., a portal or platform managed by the REMS Administrator), for example, to submit a REMS enrollment form (e.g., a prescriber may submit a patient REMS enrollment through an EHR directly to the REMS Administrator 108), and the REMS Administrator 108 communicates with the specialty pharmacies 110 and 112. All these parties (as well as other parties) are part of the fulfillment network.

The office 104 initiates the fulfillment process by preparing documents that may include a patient REMS enrollment form, a hub enrollment form (part of the applicant holder's/fulfillment partner's patient support services), and the REMS required prescription form. In the illustrated implementation, these forms may be generated using logic (software, hardware, and/or firmware) resident on computer systems of the office 104 and/or accessed via a web portal or other remote platform such as platform 106. However, it is not necessary to separately fill these forms. Rather, the office 104 can provide one set of patient information 124 that can be used to simultaneously (or sequentially) populate these forms. The patient information 124 may be provided by HCP office personnel via user devices 126, such as desktop computers, laptop computers, tablet computers, or other data devices, and may be entered locally at the office 104 or remotely. The information may be obtained from an EHR/EMR source (using an interface within or external to the EHR) or may be entered using appropriate data entry software accessed locally, via hub platform 106, or another site of the fulfillment network. These are submitted to or accessed by the hub platform 106, for example, by an EHR submission or the Online Enrollment Site (OES), to the REMS Administrator 108 for REMS patient enrollment, and to the specialty pharmacies 110 and 112. These forms, which are completed electronically, are defined by the system provider, designed to collect the information required by the various regulations and other documentation requirements, and provided in accordance with a schema.

The schema is a data structure that supports data acquisition and processing for a particular REMS, for prescription and controlled substance regulations, and/or for other programs related to patient support, insurance, and eligible financial assistance. Among other things, the schema may define fields, attributes, and values related to these objectives. The fields may relate to, for example, patient identification, drug identification, prescriber identification, and insurance provider and plan, an applicant holder, applicable patient medication and comorbidity history, and any other categories of information relevant to a prescription. Attributes may relate to, for example, the dosage, frequency, and duration of a prescription, refills, conditions or symptoms, suitability of alternative drugs, and the like. The values are generally field/attribute dependent and may be binary (yes or no/true or false), numeric, textual, or combinations thereof. The schema may further define metadata tags to describe the data (e.g., by field and attribute), data formats, messaging protocols, and other technical implementations to enable encoding, decoding and processing. Communications between the platform 106 and office 104 may be managed by a communications portal 118 including appropriate logic for managing communications in accordance with the schema and applicable regulations.

To enter patient information at the office 104, the healthcare provider or appropriate staff (collectively the Healthcare Provider or HCP) may access a prescription system with logic resident on the computer/network of the HCP and/or a remote site such as the platform 106. The logic may present the HCP with a series of logically dependent interfaces (screens, boxes, drop-down menus, etc.) organized, e.g., using branching logic, to obtain all information relevant for the REMS, controlled substance, prescription, and other documentation requirements. In addition, some of the patient information may be obtained from an EHR/EMR source, and software (e.g., using AI or other logic), may automatically recognize data fields, attributes, and values from those records. For example, the HCP may begin by entering the drug name or condition (e.g., LUMRYZ or narcolepsy) into a local prescription fulfillment application or a prescription fulfillment application of the platform 106. In response, the logic may identify prescription requirements, an applicable REMS, and REMS documentation requirements. From this, the logic can further generate a list of required information (e.g., fields, attributes, etc.) required for the fulfillment process and generate an appropriate template. Alternatively, the logic may retrieve a predefined electronic template including the required information. For example, the template may be stored in a template database and indexed to the drug, drug type, REMS, or the like. In any case, the HCP is prompted to enter the relevant information concerning the patient, drug, prescription parameters, prescriber, insurance information, and any other information relevant to the fulfillment process.

In the illustrated embodiment, the logic is implemented at least in part at the platform 106 and is accessed by the HCP using a public or private computer network, e.g., the Internet. In order to assist in generating and populating the forms, the platform is operative to access storage that includes system data 116 concerning REMS information, enrollment information, and prescription information. Although the system data 116 is illustrated in connection with the platform 106, various elements of the data 116 may reside or be accessed at the office 104, REMS Administrator 108, or distributed therebetween. The REMS information may include, among other things, information concerning what REMS program applies to which drugs, what the requirements of the REMS programs are, and what templates or forms are required. The enrollment information may include enrollment requirements, form templates, fields, attributes, and the like, of any enrollments provided under a REMS or other program. The prescription information includes any prescription requirements, including REMS prescription requirements, identification of specialty pharmacies for a prescription, and other information concerning specific prescriptions.

As the HCP navigates through the user interface elements of the submission process, the user enters values or data elements that are then associated with metadata based on the fields or attributes of the interface elements. Thus, as the HCP navigates through the user interface elements to enter prescription information for a prescription, at a certain point, the user selects the drug to be prescribed and then may be prompted, e.g., via a drop-down menu, to enter a dosage and may select the drug strength. The resulting data object of the submission may be encoded with metadata corresponding to elements of the schema as follows utilizing LUMRYZ as an example:
<LUMRYZ REMS<LUMRYZ<dosage<“7.5 g”>>>>

This indicates that the entry “7.5 g” is a dosage of LUMRYZ which has a LUMRYZ REMS.

Similarly, when other information is entered into the system 100, it may be associated with metadata according to the schema. Thus, a pharmacy named “XYZ Pharmacy” may be encoded as follows.
<LUMRYZ REMS<specialty pharmacy< “XYZ Pharmacy”>>>

This indicates that the “XYZ Pharmacy” is a specialty pharmacy certified for fulfillment in the LUMRYZ REMS.

The platform 106 may also be operative to access insurance and prescription assistance information. The insurance information may include information regarding coverage, co-pays, and prescription limitations from various insurers and plans. The prescription assistance information may include information concerning programs to help certain patients obtain free or low-cost prescriptions or other financial assistance. The platform 106 may be connected to platforms of the insurance providers 120 and prescription assistance programs 122 to obtain an update of the information.

In operation, the HCP accesses the platform 106 to initiate the fulfillment process. The HCP enters information indicating the desired prescription. Based on this information, the controller 114 (e.g., one or more computers or servers) of the platform 106 executes the fulfillment logic and accesses storage 116 to identify the REMS, controlled substance, prescription, and other documentation requirements implicated, identifies the information required, and/or accesses a template for obtaining the required information. This is used to provide to the HCP a template or form for prompting the HCP to enter the information required for fulfillment.

Next, this information is used to populate the forms required for fulfillment. For example, based on the relevant REMS, the platform 106 may identify, to the HCP office 104, the specialty pharmacy (or identify the specialty pharmacies from which the HCP may select) that is in the patient's insurance network for the prescription to be submitted to. The controlled substance prescription regulations generally require that the electronic prescription is transmitted directly from the HCP office 104 to the pharmacy 110 or 112. A separate REMS prescription form may also be required. Thus, the platform 106 may provide an appropriate REMS prescription form or other prescription information to the office 104 for submission by the office 104. It will be appreciated that various relationships between the illustrated parties and communications pathways are possible depending on the implementation and applicable REMS. For example, while the prescription must generally be provided from the HCP office 104 to the pharmacy 110 or 112, there may be possible implementations where, for example, the platform 106, which is not limited to a hub, acts on behalf of a specialty pharmacy, e.g., a non-commercial specialty pharmacy, or provides the means by which the REMS prescription form is submitted to the specialty pharmacy.

In addition, the controller 114 of the platform 106 may access system data 116 to generate a packet of information or populate forms for transmission (or otherwise provided) from the platform 106 to the specialty pharmacy 110 or 112, from the REMS Administrator 108 to the specialty pharmacy 110 or 112, and/or from the office 104 to the REMS Administrator 108. The REMS may specify that the pharmacy 110 or 112 needs to have confirmation of various items before a prescription can be filled, e.g., relating to verifying required disclosures to patients and HCPs, program enrollments, or confirming monitoring requirements or drug interaction notices. In some cases, the pharmacy 110 or 112 may access a platform to obtain the required information. For example, the pharmacy may access the REMS Administrator 108 to validate enrollment. The packets may include documentation required by the pharmacy 110 or 112, the REMS, controlled substance, and prescription regulations, other than the prescription that is obtained directly from the office 104. Later, if the prescription is moved from pharmacy 110 to pharmacy 112, the packet or relevant portion thereof can conveniently be provided to the new pharmacy 112 and a prescription form can be provided by the platform 106 to the office 104. The office 104 can then provide an appropriate prescription to the new pharmacy 112.

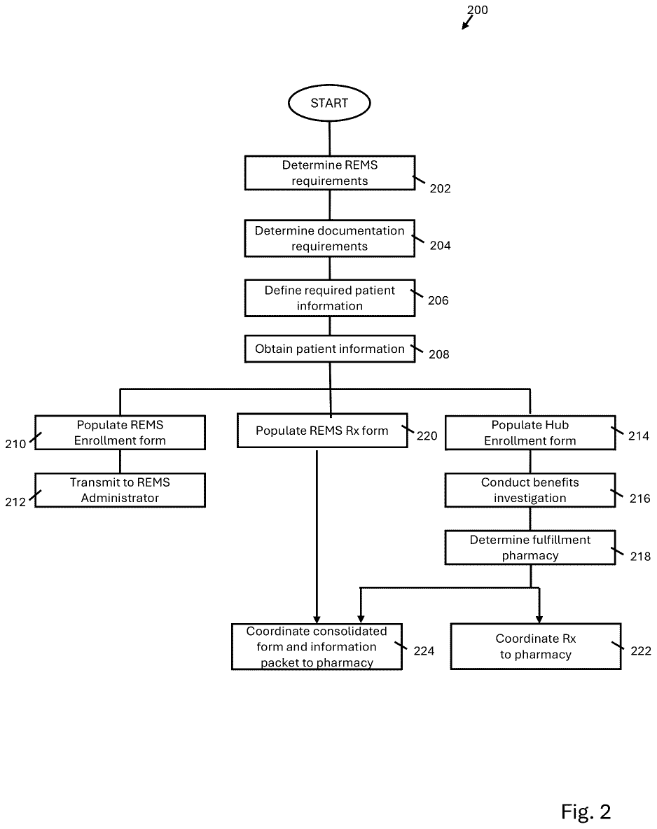

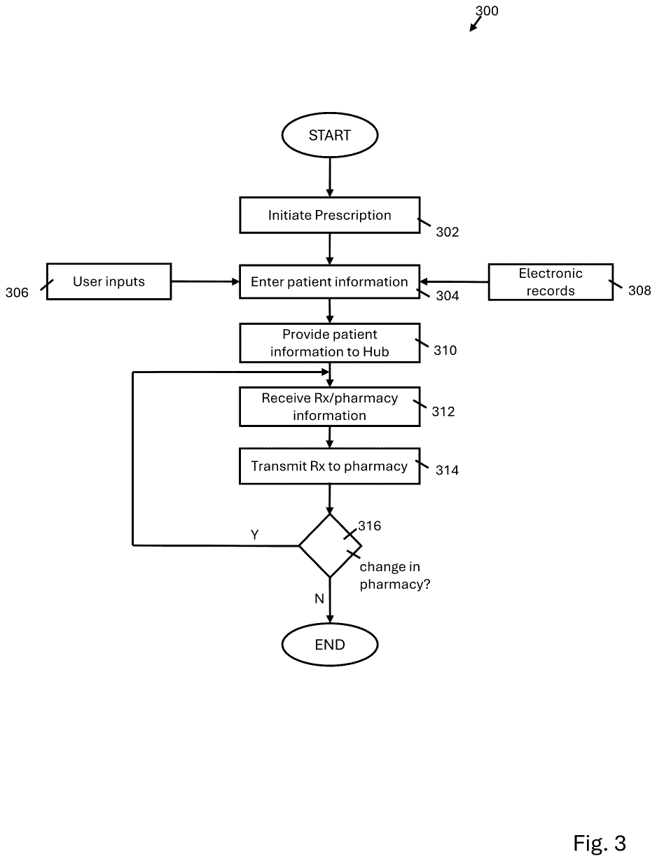

This process may be summarized by reference to the flowchart of FIGS. 2-3. FIG. 2 illustrates a process associated with a fulfillment system of the present invention. The process 200 is initiated by determining (202) REMS requirements associated with a prescription fulfillment. For example, an HCP may initiate a prescription by accessing the fulfillment system (e.g., via a portal, or logic residing on a computer network of the HCP) and entering information identifying the drug to be prescribed. Based on the drug being prescribed, the system may access system data to identify any REMS associated with the drug and the requirements of the REMS. From the REMS and other sources of information, the system may determine (204) the documentation requirements for the prescription, e.g., hub and REMS enrollment, prescription form requirements, etc. Based on these documentation requirements, the system can provide a template, a series of user interfaces, or other prompts to obtain (208) the patient information for fulfilling the documentation requirements. That is, the system determines the set of information required in connection with the required forms and prompts the HCP to enter that patient information. In the example of fulfilling a LUMRYZ prescription, the office accesses the platform and initiates the LUMRYZ fulfillment process. The platform provides a template that is tailored to obtain all the information required under the relevant REMS, prescription, controlled substance, and other documentation requirements applicable for that prescription.

The platform uses this information to populate (210, 214, and 220) a REMS enrollment form, patient support services or hub enrollment form, and the appropriate REMS prescription form, and any other required documentation. In the case of the REMS enrollment form, the form may then be transmitted (212) from the HCP to the REMS Administrator. The hub enrollment form may be transmitted to and/or retained at the hub. The hub may then conduct (216) a benefits investigation to determine insurance coverage information (e.g., coverage availability, copay, prescription limits, etc.) and educate on (218) an appropriate fulfillment pharmacy. As noted above, different payors may have relationships with different specialty pharmacies. The system can then coordinate (224) the provision of the documentation as required. For example, a consolidated form and information packet may be provided from the hub to the fulfillment pharmacy. In some cases, when the pharmacy is processing a prescription, the pharmacy accesses the REMS Administrator platform to verify enrollment. Thus, the information required by the pharmacy may be obtained from multiple sources and may be pushed to the pharmacy and/or pulled from the hub and REMS Administrator.

The system may also populate a REMS prescription form for the HCP to submit to the pharmacy. Additionally, e.g., when required by state law, the system may also populate an electronic prescription (also known as eScript) for the HCP. In this manner, the system may coordinate (222) with the HCP to submit the prescription as required. In the case of a LUMRYZ prescription, this process supports multiple specialty pharmacies of the LUMRYZ fulfillment network as well as pharmacies handling free or reduced-price prescriptions, e.g., under a Prescription Assistance Program (PAP). The platform further operates as a repository maintaining copies of the documentation should any further prescription assistance needs arise for eligible patients.

In connection with the benefits investigation, the system may verify a patient's insurance coverage and locate applicable prior authorization documentation for the HCP as needed. Once a payer determination is made, if the case is approved, the system may notify the HCP via fax or other modality which in-network pharmacy could be servicing the patient. The HCP will then send a valid prescription (eScript, fax, verbal) to the servicing in-network pharmacy. The system can then send the packet to the servicing pharmacy including, for example, a cover sheet with identification information and any other required information. In the event of a change in pharmacy, the packet can be sent to the new pharmacy, and a valid REMS prescription form can be provided to the new pharmacy for fulfillment. The provider of the platform may also designate a specialist or hub services to guide the patient through the fulfillment process.

FIG. 3 is a flowchart illustrating a fulfillment process 300 associated with an HCP. In the illustrated process, the HCP initiates (302) a prescription by accessing the fulfillment system and identifying the drug to be prescribed. It will be appreciated that the prescription process may vary in certain contexts such as prescription renewals. In the illustrated example, the system prompts the HCP to enter (304) the required patient information. For example, the HCP may be provided with a template including interface elements for entering the items of patient information required for the prescription process (e.g., relating to hub and REMS enrollments, insurance, prescription form requirements, etc.). The patient information may be entered by user inputs (306) into the template and/or by importing data from an EHR/EMR system (308).

In the illustrated implementation, the patient information is provided (310) to a hub that, among other things, conducts a benefits investigation to determine coverage and identify an appropriate specialty pharmacy. The HCP then receives (312) information from the hub identifying the appropriate specialty pharmacy and otherwise providing information for use by the HCP in submitting a prescription. This may include a REMS prescription form. The HCP can then transmit (314) a prescription, e.g., an eScript, to the identified specialty pharmacy. Later, if the specialty pharmacy changes (316), the HCP can receive new pharmacy information and transmit a prescription to the new pharmacy.

As described herein, the various offices and other platforms communicate with the hub platform to implement a variety of functions. Although the hub platform is illustrated as a single element, it will be appreciated that the platform may be executed on one or more machines (e.g., computers or servers) at a single site or geographically distributed. Each such site may execute the full functionality of the illustrated platform, or the functionality may be distributed across sites. Moreover, the functionality may be distributed in various ways between the hub platform, the offices, and other platforms, e.g., some preprocessing of prescription information or other information may be executed at the offices or other platforms, for example, to facilitate rapid response or reduce use of processing resources of the hub platform or communication bandwidth requirements. The hub platform may be hosted by a system provider or may be implemented separately (e.g., cloud-based) and connected to the system provider via an interface such as application programming interface (API). Moreover, the hub platform may be hosted by an office or other platform among other possibilities.

Referring again to FIG. 1, although that Figure shows, for purposes of illustration, a single patient 102 and a single office 104, it will be appreciated that the platform 106 can service many patients 102 of many offices 104. Similarly, although two specialty pharmacies 110 and 112 are shown, more specialty pharmacies may be available for a given REMS and different pharmacies may be involved in other fulfillment processes. There may also be more insurance 120 and prescription assistance 122 platforms and other platforms that may be connected to the hub platform 106.

The handling and communication of healthcare information is subject to significant regulations concerning privacy, the mode of communication, the content of communications, and the authorized recipients. The platform 106 can use certain industry-standard infrastructure, implement compliant security measures, and format/filter communications in compliance with these regulations and requirements. In this regard, the system may make use of existing standards including eScript, defined personal health record (PHR) standards, and OES for documentation and transmissions. Moreover, information handling and transmission may be routed via appropriate electronic means, monitored and filtered as necessary for content, and otherwise handled to be compliant with HIPAA.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art are within the scope of the present invention. The embodiments described herein above are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A method for facilitating fulfillment of prescriptions for regulated drugs, comprising:
operating a computer network platform including a communications port for implementing electronic communications in compliance with applicable regulations concerning electronic transmission of healthcare related information, wherein the operating comprises:
first obtaining documentation information relating to a first fulfillment of a first prescription of a first patient for a first drug, said documentation information indicating document requirements concerning at least first and second parties involved in said first fulfillment;
second obtaining patient information concerning said first patient;
accessing a schema relating to said documentation requirements, said schema defining data, including defined fields of information concerning said first prescription, for use in populating documents associated with said documentation requirements;
first extracting, from said patient information, first data corresponding to a first set of said defined fields of information concerning said first prescription;
first electronically populating, using said first data, first data objects of a first document of said documents to provide a first populated document, said first populated document at least partially satisfying a first documentation requirement of said documentation requirements;
first providing said first populated document to a first party involved in said first fulfillment;
second extracting, from said patient information, second data corresponding to a second set of said defined fields of information, the same as or different than said first set of said defined fields of information, concerning said first prescription;
second electronically populating, using said second data, second data objects of a second document of said documents to provide a second populated document, said second populated document at least partially satisfying a second documentation requirement of said documentation requirements; and
second providing said second populated document to a second party involved in said first fulfillment;
wherein said first and second populated documents facilitate fulfillment of said prescription in compliance with said first and second documentation requirements.

2. The method of claim 1, further comprising entering said patient information via a computer system of a healthcare provider.

3. The method of claim 2, wherein said patient information is received at said computer network platform via an electronic communication between said computer system of said healthcare provider and said computer network platform in compliance with said applicable regulations concerning electronic transmission of said healthcare related information.

4. The method of claim 2, wherein said patient information is entered on said computer system using an electronic template that prompts a user to enter information items for a plurality of said defined fields of information concerning said prescription and is operative for associating metadata concerning said defined fields of information with said information items.

5. The method of claim 4, wherein said information items and said metadata are used in said first and second extracting.

6. The method of claim 1, wherein said computer network platform comprises logic, executed on a processor, for controlling transmissions in accordance with requirements under one or more of the United States Health Insurance Portability and Accountability Act (HIPAA) and other regulations concerning handling of healthcare related information.

7. The method of claim 1, wherein one of said first and second populated documents comprises an enrollment form.

8. The method of claim 7, wherein said enrollment form comprises one of a Risk Evaluation and Mitigation Strategy (REMS) enrollment form, a hub enrollment form, and a patient support services enrollment form.

9. The method of claim 1, wherein one of said first and second populated electronic documents comprises a prescription form.

10. The method of claim 9, wherein said prescription form comprises a form required under a Risk Evaluation and Mitigation Strategy (REMS).

11. The method of claim 1, wherein one of said first and second populated documents comprises information identifying a pharmacy for fulfillment of said prescription.

12. The method of claim 1, wherein said first party comprises one of a healthcare provider and a pharmacy.

13. The method of claim 12, wherein said pharmacy comprises a certified pharmacy under a REMS.

14. The method of claim 12, wherein said second party comprises one of a REMS Administrator and a hub.

15. The method of claim 1, wherein said first party comprises a one of a payor and a prescription services entity.

16. The method of claim 1, further comprising storing said patient information, receiving an indication of a change to a new pharmacy, and third providing a third populated document to said new pharmacy.

17. The method of claim 1, wherein said communications port comprises logic for controlling communications between said computer network platform and external platforms.

18. A system for facilitating fulfillment of prescriptions for regulated drugs, comprising:
a computer network platform including a communications port for use in transmitting electronic communications in compliance with applicable regulations concerning electronic transmission of healthcare related information;
said computer network platform being operative for:
receiving patient information including data corresponding to defined fields of information concerning a prescription;
first extracting, from said patient information, first data corresponding to a first set of said defined fields of information concerning said prescription;
first electronically populating, using said first data, first data objects of a first document to provide a first populated document, said first populated document at least partially satisfying a documentation requirement pertaining to said prescription;
first providing said first populated document to a first party involved in fulfillment of said prescription;
second extracting, from said patient information, second data corresponding to a second set of said defined fields of information, the same as or different than said first set of said defined fields of information, concerning said prescription;
second electronically populating, using said second data, second data objects of a second document to provide a second populated document, said second populated document at least partially satisfying a second documentation requirement pertaining to said prescription; and
second transmitting said second populated document to a second party, the same as or different than said first party, involved in fulfillment of said prescription;
wherein said first and second populated documents enable fulfillment of said prescription in compliance with said first and second documentation requirements.

19. The system of claim 17, further comprising a computer system of a healthcare provider operative for entering said patient information.

20. The system of claim 18, wherein said patient information is received at said computer network platform via an electronic communication between said computer system of said healthcare provider and said computer network platform in compliance with said applicable regulations concerning electronic transmission of said healthcare related information.

21. The system of claim 18, wherein said patient information is entered on said computer system using an electronic template that prompts a user to enter information items for a plurality of said defined fields of information concerning said prescription and is operative for associating metadata concerning said defined fields of information with said information items.

22. The system of claim 20, wherein said information items and said metadata are used in said first and second extracting.

23. The system of claim 17, wherein said computer network platform comprises logic, executed on a processor, for controlling transmissions in accordance with requirements under one or more of HIPAA and other regulations concerning handling of healthcare related information.

24. The system of claim 17, wherein one of said first and second populated documents comprises a patient support services enrollment form.

25. The system of claim 17, wherein one of said first and second populated documents comprises a prescription form.

26. The system of claim 25, wherein said prescription form comprises a form required under a Risk Evaluation and Mitigation Strategy (REMS).

27. The system of claim 17, wherein one of said first and second populated documents comprises information identifying a source pharmacy for fulfillment of said prescription.

28. The system of claim 17, wherein one of said first and second parties comprises a pharmacy.

29. The system of claim 27, wherein said source pharmacy comprises a certified pharmacy under a Risk Evaluation and Mitigation Strategy (REMS).

30. The system of claim 17, wherein one of said first and second parties comprises a healthcare provider.

31. The system of claim 17, wherein one of said first and second parties comprises a prescription assistance program entity.

32. The system of claim 17, wherein said computer network platform is further operative for storing said patient information, receiving an indication of a change to a new pharmacy, and third providing a third populated document to said new pharmacy.

33. The system of claim 17, wherein said communications port comprises logic for controlling communications between said computer network platform and external platforms.
